(19) United States
(12) Patent Application Publication
(10) Pub. No.: US 2021/0100654 A1
(43) Pub. Date: Apr. 8, 2021

(54) Catheter Sheath Control Wire

(71) Applicants: Michael Ring, Spokane, WA (US); Paul Knight, Spokane, WA (US)

(72) Inventors: Michael Ring, Spokane, WA (US); Paul Knight, Spokane, WA (US)

(21) Appl. No.: 17/063,896

(22) Filed: Oct. 6, 2020

Related U.S. Application Data
(60) Provisional application No. 62/912,057, filed on Oct. 7, 2019.

(51) Int. Cl. A61F 2/24; A61M 25/09; A61M 25/01

(52) U.S. Cl. CPC A61F 2/2436; A61M 25/0147; A61M 25/09

(57) ABSTRACT

A transcatheter delivery device having a sheath control wire and methods of use are described herein. A novel method for optimizing the angle between a capsule having a prosthetic heart and the catheter sheath is described. The catheter sheath contains a sheath wire in connection with an actuator of a delivery device. The sheath wire has a straight section and an angled section which cause the catheter sheath to bend at a flexible section.

### CROSS REFERENCE TO RELATED APPLICATION

This application claims the benefit of pending U.S. provisional application Ser. No. 62/912,057 filed Oct. 7, 2019 by the present inventors, which is incorporated by reference in its entirety.

### STATEMENT REGARDING FEDERALLY SPONSORED R&D

Not related to this application.

### TECHNICAL FIELD

This invention relates to catheter delivery systems and more particularly to, but not limited to, catheter delivery systems used in prosthetic heart value replacement procedures.

### BACKGROUND OF THE INVENTION

Heart valve replacement is the process of replacing a natural heart valve with a prosthetic valve. The prosthetic valve is typically used in transcatheter procedures and is comprised of valve leaflets attached within an expending metal mesh, inflatable stent, or other structure. The valve assembly is contained within a capsule of a delivery system prior to deployment. At the desired location within the heart, the capsule is retracted to allow the valve, or valve stent, to expand and be secured to the outer walls of the aortic annulus. In devices with balloon expandible stent valves, an external indeflator is used to inflate the balloon. Regardless of the mechanism, heart valve replacement requires the prosthetic valve to displace the natural valve, which requires the capsule to be located precisely and accurately within a beating heart.

In more detail, the heart valve replacement process starts by a medical professional loading or crimping a prosthetic heart valve to the capsule that is connected to a delivery catheter sheath. The catheter sheath is a hollow tube, often plastic reinforced with steel banding, that extends from the capsule to a delivery device. Within the catheter sheath is typically a hollow tube which accommodates a guide wire. Once the capsule is loaded with the valve stent, the medical professional introduces the catheter sheath into the vasculature via a guide wire that was previously navigated through the tortuous vessels of the body and into the left ventricle of the heart. The guide wire is typically thin and flexible in comparison to the catheter sheath of the deployment device system. Once the guide wire is optimally located in the heart, the medical professional slides the catheter sheath over the guide wire and begins to slide the capsule and catheter sheath along the guide wire. The stiffer catheter sheath utilizes the guide wire to help it navigate bends, such as the aortic arch. Utilizing scanning and video technology, the medical professional moves the guide wire and catheter sheath relative to the patient's anatomy to optimally locate the capsule relative to the natural valve and surrounding anatomy. The medical professional may repeatedly push, pull and twist the deployment device in hopes to achieve a more optimal position to start the prosthetic valve deployment with varying success. Movement of the catheter sheath, capsule and guide wire creates a risk of damaging the surrounding tissue. However, a sub optimally positioned replacement valve may lead to a diminished valve function and/or complications. Once the valve position appears to be optimally located relative to the unique anatomy of each individual patient, the medical professional activates the deployment device system to cause the artificial valve to deploy. Once deployed, the valve is no longer removable by the delivery device. An invasive surgical procedure may be necessary to reposition or remove a deployed valve. Surgical intervention of the prosthetic valve is not always an option for these patients. It should be appreciated that the medical professional performing the prior art heart valve replacement procedure must have the experience, judgment and dexterity to precisely locate the artificial valve within the unique geometry of an individual patient's heart.

A potential weakness of prior art delivery devices is that they are focused on managing axial movement of the capsule along the length of the aorta arch and annulus. As the stiff catheter sheath is redirected by the resistance of the tissue of the aortic arch, the catheter sheath forces the capsule towards the outer edge of the annulus. This position causes the deployed valve to be canted or angled relative to the natural valve and annulus region. U.S. Pat. Nos. 9,918,838 and 9,919,130, also to inventor Michael Ring, describe a method of helping position a guide wire in an ideal coplanar and coaxial orientation which impacts prosthetic valve deployment. The patents also describe guide wire shapes that can alter the angle of the capsule relative to the catheter sheath. A potential drawback to the prior art is that managing both the position of the delivery catheter/capsule and the guide wire must be accomplished with the same motion. Another potential drawback of the current art is that a guide wire alone may not have enough stiffness to deflect stiff catheters sheaths.

In these respects, the present invention departs from conventional concepts of the prior art by providing a parallel (to the guide wire) catheter sheath wire control device for use in catheter based medical procedures. The present invention also provides an improved way to achieve optimal valve deployment in transcatheter valve replacement and repair procedures, as well as any medical procedure requiring additional control beyond that afforded by the guide wire alone.

### SUMMARY OF THE INVENTION

The present invention takes a very different approach to controlling the position of the delivery catheter while deploying a transcatheter prosthetic heart valve in comparison to the prior art.

The present invention provides a device for controlling the angle of a capsule relative to the catheter sheath during a surgical procedure. The variable angle of the catheter sheath relative to the capsule allows for more optimal deployment of transcatheter deployed medical device.

A sheath wire has a straight section, a bend, and an angled section. The straight section is retained by an actuator as part of a delivery device assembly. The bend of the sheath wire is in close proximity to a novel pivot point of the sheath. The pivot point may be formed by a reduction in outer diameter or a change in material properties of the catheter sheath relative to the rest of the catheter sheath. The result is that the bend of the sheath wire as it translates down the length of the sheath causes the catheter sheath in front of the pivot and capsule to bend relative the sheath section behind the pivot point.

A first object of the present invention is to allow a medical professional to change the angle of a deployment capsule relative to the axis of a valve annulus within the heart.

A second object of the present invention is to enable the movement of the deployment capsule relative to the axis of the aortic annulus without moving a deployed guidewire.

A third object of the present invention is to enable the movement of a deployment capsule through the use of a catheter sheath wire having straight section, bend, and an angled section.

Control of a sheath wire, according to the present invention, provides the advantages of providing better locational accuracy of catheter delivered medical devices. The preferred embodiments for both the apparatus and process is described for use in heart valve repair and replacements, but the present invention is applicable to any medical procedure utilizing a catheter.

These and other features, aspects, and advantages of the present invention will become better understood with regard to the following description, appended claims, and accompanying drawings.

### BRIEF DESCRIPTION OF THE DRAWINGS

Preferred embodiments of the invention are described below with the reference to the following accompanying drawings:

FIG. 1 is a front partial section view of a heart with a prior art guide wire inserted through the aortic artery and into the left ventricle of the heart.

FIG. 2 is the same front partial view as FIG. 1, but with a prior art catheter sheath and an artificial valve inserted around the guide wire of FIG. 1 and into the heart. A capsule of the catheter assembly is shown undesirably uncentered and off-angle in the aorta.

FIG. 3 is the same front partial section view of FIG. 1 and showing an optimally deployed and centered artificial valve.

FIG. 4 is a perspective view showing a deployment end of a novel heart valve deployment system according to the present invention.

FIG. 5 is a top view of an actuation end of the novel delivery system according to the present invention.

FIG. 6 is a side view of a novel sheath control wire which travels through the deployment system of FIG. 4 and FIG. 5.

FIG. 7 is a detail view of the shaped end of the novel sheath wire control wire of FIG. 6.

FIG. 8 is a front partial section view of a partially deployed heart valve in use with the novel deployment system according to the present invention.

FIG. 9 is cross section view of the catheter sheath of the present invention showing the guide wire and sheath wire inside the sheath.

FIG. 10 is a cross section view of an alternative embodiment catheter sheath of the present invention wherein the guide wire and sheath wire are separately captured within the catheter sheath.

FIG. 11 is an alternative embodiment sheath of the present invention and showing an oval inner catheter sheath tube and oval sheath wire.

FIG. 12 is a perspective view of a complex shaped alternative embodiment of the sheath wire.

FIG. 13 is a flow diagram for a novel process and method for deploying an artificial heart valve according to the present invention.

### DETAILED DESCRIPTION OF THE PREFERRED EMBODIMENTS

Many of the fastening, connection, wiring, control, manufacturing and other means and components utilized in this invention are widely known and used in the field of the invention, and their exact nature or type is not necessary for a person of ordinary skill in the art or science to understand the invention; therefore they will not be discussed in detail. Furthermore, the various components shown or described herein for any specific application of this invention can be varied or altered and anticipated by this invention and the practice of a specific application or embodiment of any element may already be widely known or used in the art, or persons skilled in the art or science; therefore, each will not be discussed in significant detail.

The present invention, as described, is a novel catheter sheath and sheath wire as part of a catheter assembly for use during medical procedures. Due to catheter sheaths typically being more rigid than guide wires, a guide wire is first used to navigate tortuous pathways of the body and then the guide wire is inserted into the sheath allowing the sheath to travel the pathway of the guide wire. Although the present invention is primarily described for use with a catheter within an aortic artery, it should be appreciated that the present invention should not be construed to be limited to any particular body lumen. Other applicable lumens include, but are not limited to, gastrointestinal and urine lumens. Similarly, the present invention is primarily described for use with heart valve replacement procedures, but the present invention should not be construed to be limited to any particular procedure. Other applicable procedures include, but are not limited to, coronary angioplasty, stenting procedures and angiograms. The present invention is applicable to any pathway that a sheath is too stiff to easily navigate a lumen even when around a guide wire. The present invention is applicable to medical devices that rely on the positioning of a device within a lumen and wherein the stiffness of the sheath hinders optimal placement of medical devices.

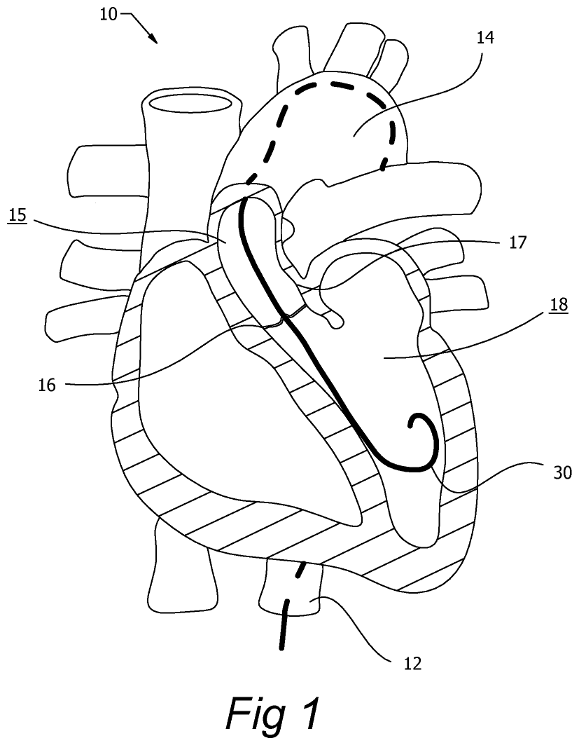

Now referring to the figures, FIGS. 1, 2 and 3 show a partial section view of a heart 10. The anatomy of heart 10 is well known in the art of medicine and a detailed understanding is not necessary for one to understand and appreciate the present invention; therefore it will not be described in significant detail. Components of heart 10 shown in the accompanying drawings are in the non-limiting context of using the present invention in an aortic valve replacement procedure.

In replacing an aortic valve and referring to FIG. 1, a guide wire 30 is advanced through an aortic artery 12, through a natural aortic valve 16, and into a left ventricle 18. Aortic artery 12 starts in the abdomen. An aortic arch section 14 comes from the back side of the heart and bends towards an ascending aorta section 15 which is just before aortic valve 16. Blood leaving left ventricle 18 escapes through natural aortic valve 16. Aortic valve 16 is surrounded by an aortic valve annulus section 17. It should be appreciated that the lumens of heart 10 are complex in shape and trajectory.

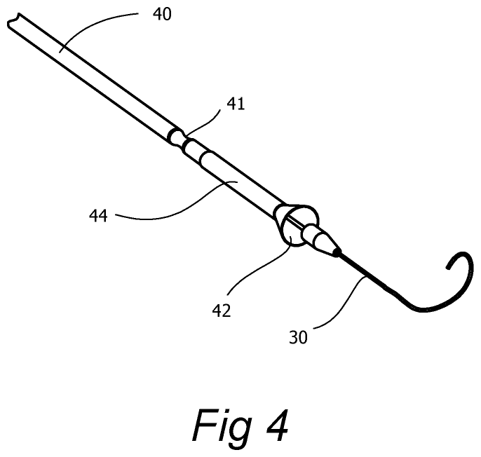

FIG. 4 shows the distal end of a catheter sheath 40 for use with a heart valve replacement delivery system 50. A guide wire 30 is approximately 0.035 inches in diameter and made from a metallic material which is coated in a low friction material, such as polytetrafluorethylene. Catheter sheath 40 is advanced over guide wire 30. Catheter sheath 40 is connected to a capsule 44 which houses a prosthetic valve 42. In FIG. 4, prosthetic valve 42 is shown in a partially deployed state. With advancement of catheter sheath 40, prosthetic valve 42 is completely encapsulated within capsule 44. With retraction of catheter sheath 40, prosthetic valve 42 is deployed. It should be appreciated that capsule 44 and sheath 40 are more typically more rigid than guide wire 30.

The application delivery system 50 of FIG. 4 is shown in FIGS. 1, 2 and 3. In FIG. 1, guide wire 30 has been advanced through aorta 12, has navigated both aortic arch 14 and ascending aorta 15 sections, has penetrated though natural valve 16, and has the guide wire distal end located within left ventricle 18. It should be appreciated at the stage of FIG. 1, the surgeon has advanced guide wire 30 by applying forces to the proximal end of guide wire 30. Imaging and feel ensures guide wire 30 is properly placed in heart 10. Guide wire 30, when placed in heart 10, has some impact to the normal function of heart 10. Therefore, it is desirable for the surgeon to act quickly and precisely to deploy prosthetic valve 42.

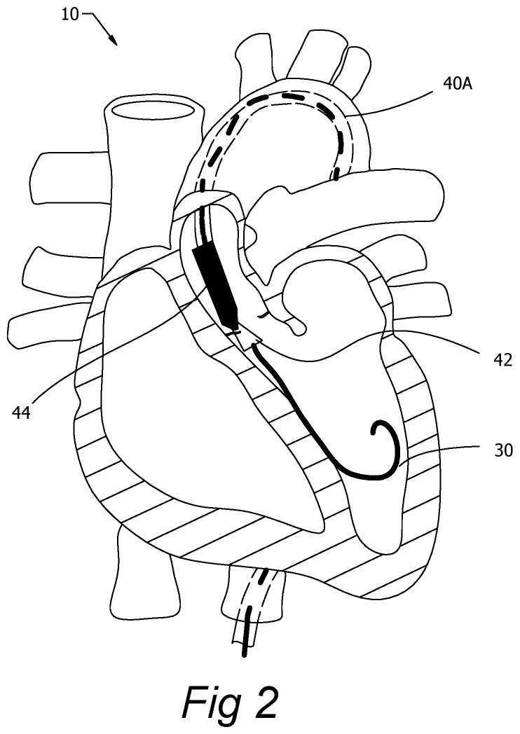

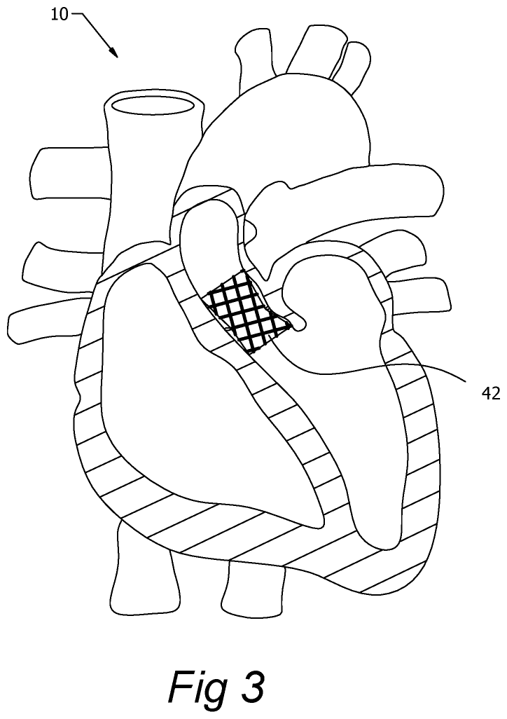

FIG. 2 shows a prior art catheter sheath 40A advanced over and along guide wire 30. Optimal location of prosthetic valve 42 in relationship to natural valve 16 and aortic annulus section 17 may be plus or minus one to three millimeters. Once optimal location of prosthetic valve 42 has been achieved both radially and in depth, the surgeon retracts prior art catheter sheath 40A causing deployment of prosthetic valve 42. The view of FIG. 2 describes a prior art deployment device wherein capsule 44 is shown off center to the axis of section 17. This situation is to be avoided as a better patient result can be achieved by aligning valve 42 to the axis of heart section 17, such as shown by FIG. 3. As FIG. 2 shows, misalignment is caused by the sheath and catheter being too stiff to properly follow the lumen of the aorta.

As shown in FIG. 4, novel catheter sheath 40 has a flexible section 41 which is located between capsule 44 and deployment system 50. Flexible section 41 provides a pivot point, or location of length, with either a reduced diameter or made of more flexible materials than the rest of catheter sheath 40, that allows capsule 44 to more easily bend with respect to catheter sheath 40. As will be described further, a novel sheath wire 70 provides control over the bend of capsule 44 with respect to sheath 40 and enables valve 42 to be aligned to the axis of heart annulus section 17. As a non-limiting example, flexible section 41 may be less than half the diameter of the rest of catheter sheath 40.

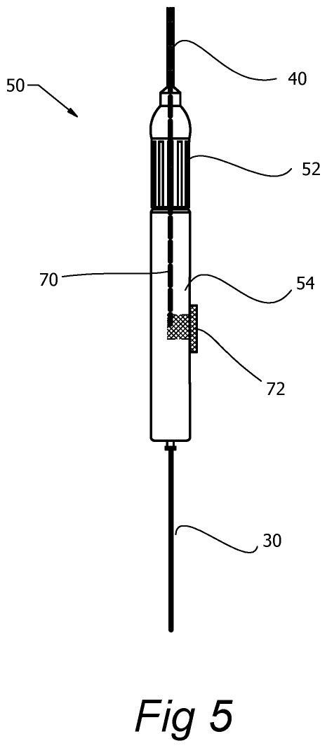

FIG. 5 shows the actuator end of deployment system 50 which controls the movement of sheath 40 and deployment of prosthetic valve 42. The surgeon holds heart valve deployment device 50 via deployment device handle 54. Prosthetic valve 42 is deployed by turning a deployment actuator 52 relative to deployment device handle 54. Guide wire 30 translates through and exits the end of deployment system 50. A novel sheath actuator 72 is rigidly connected to a straight section 74 of sheath wire 70. A surgeon moving sheath actuator 72 forward (towards the body) moves sheath wire 70 forward relative to catheter sheath 40. Sheath wire 70 remains within sheath 40. Sheath wire 70 is preferably made from a metallic wire but is not limited to such. Any material may be used to construct sheath wire 70 within the spirit and scope of the present invention.

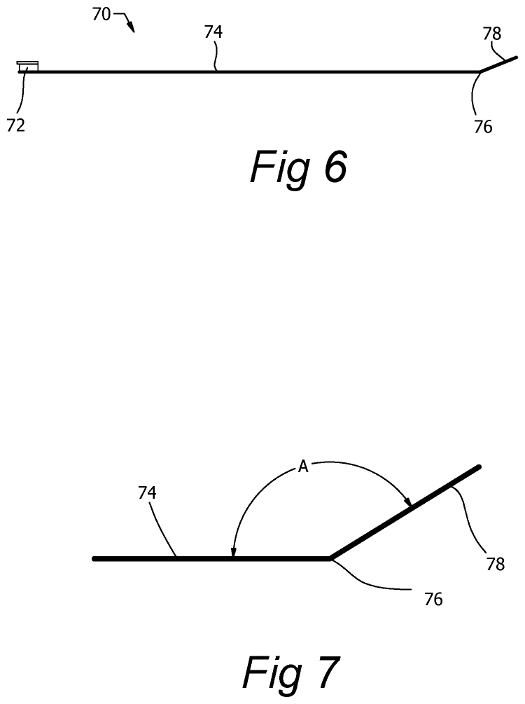

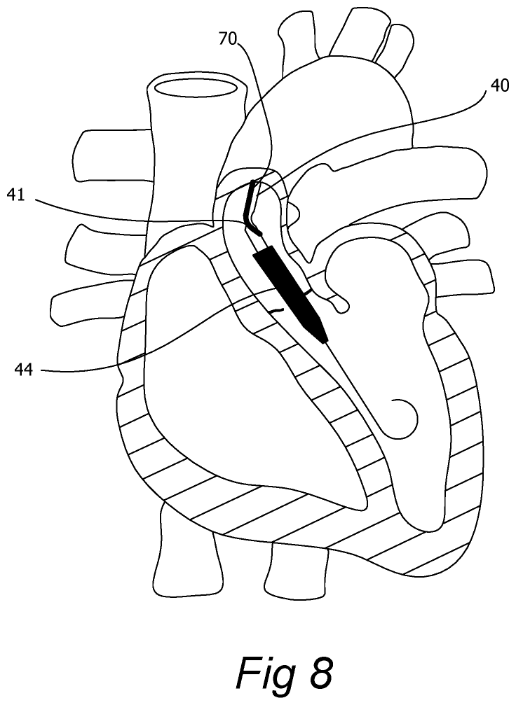

FIG. 6 shows a side view of sheath wire 70. Actuator 72 is attached to one end of a straight sheath wire section 74. At the other end is at least one bend 76 and an angle section 78. It should be appreciated that an angle A is formed between straight section 74 and angle section 78. FIG. 8 shows sheath wire 70 in the actuated position wherein angle section 78 in combination with sheath flexible section 41 causes capsule 44 to bend with respect to catheter sheath 40. Angle section 78 in combination with sheath flexible section 41 allows capsule 44, and ultimately valve 42, to be aligned to the axis of heart section 17. The result is better deployment of valve 42. When bend 76 is not in close proximity, or not within, flexible section 41, capsule 44 remains aligned to the end of catheter sheath 40. When bend 76 is in close proximity, or within, flexible section 41, capsule 44 will bend at an angle relative to catheter sheath 40. It should be appreciated that sheath wire 70 is attached to actuator 72, in a fixed manner by means of glue, adhesive or fasteners, and that the other end is allowed to slide in the axial direction, both forward and back, relative to catheter sheath 40. Straight section 74 within catheter sheath 40, bend 76 in close proximity or within flexible section 41 and angled section 78 in contact with either catheter sheath 40 or capsule 44 causes capsule 44 to move relative to catheter sheath 40.

### Use

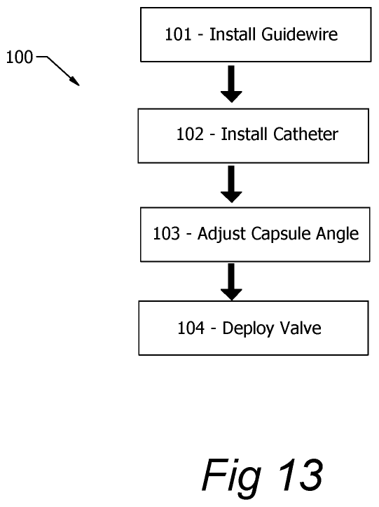

FIG. 13 shows a method 100 for using deployment system 50 for deploying valve 42 within heart 10. The surgeon installs guide wire 30 through the lumens of the body and into heart 10 utilizing a step 101. Once optimal location of guide wire 30 is obtained, catheter sheath 40 is traversed along guide wire 30 by inserting guide wire within catheter sheath 40 utilizing a step 102. Actuator 70 is unactuated and angled section 78 is not in close proximity to, or not within, flexible section 41. When capsule 44 is at the right distance into heart 10, the surgeon performs a step 103 by moving actuator 72 causing angled section 78 to be in close proximity to flexible section 41 and for capsule 44 to bend with respect to catheter sheath 40, and to optimally located valve 42 within heart 10. When optimal location of capsule 44 is obtained, the surgeon performs a deploy step 104. Step 104 has been previously described with retracting catheter sheath 40 causing deployment of valve 42.

### Alternative Embodiments

Although the preceding descriptions set forth the best mode of the present invention there are numerous alternative embodiments that all fall within the spirit and scope of the present invention.

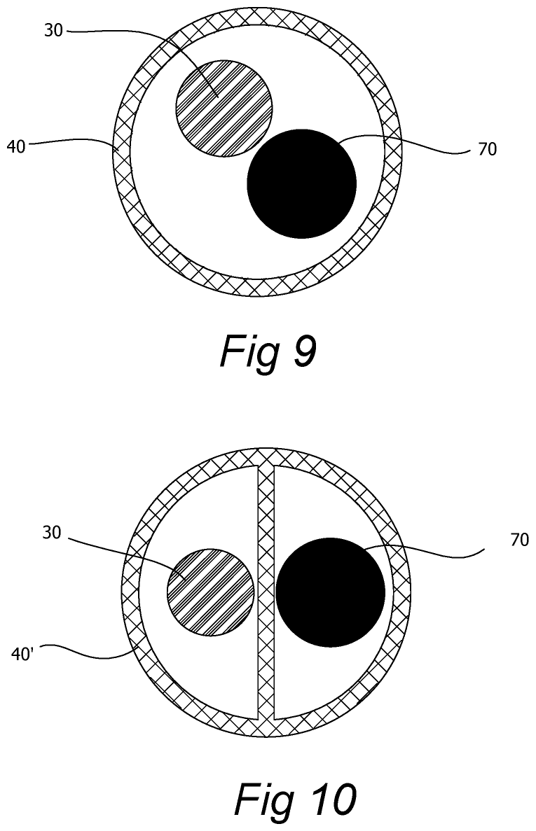

As shown in the cross section view of FIG. 9, the best mode of the invention has guide wire 30 and sheath wire 70 within catheter sheath 40 and sharing space.

FIG. 10 shows an alternative embodiment wherein catheter sheath 40′ has two channels with one capturing guide wire 30 and the other capturing sheath wire 70.

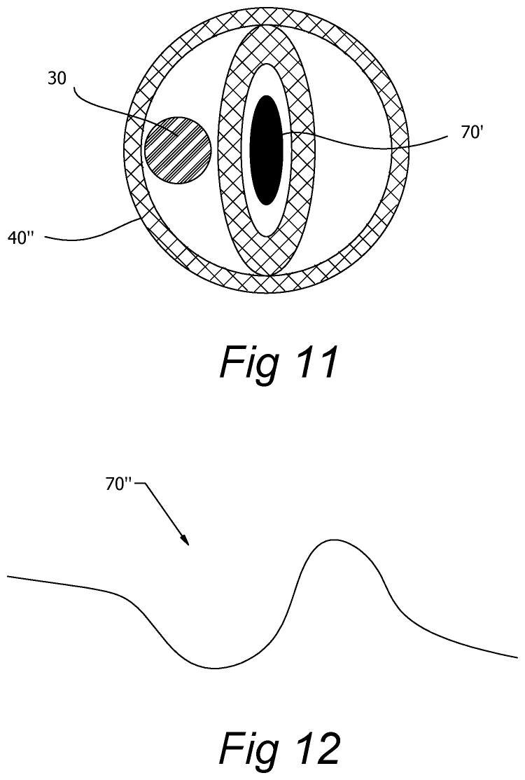

FIG. 11 shows yet another embodiment that has guide wire 30 within a channel of a catheter sheath 40″ and sheath 40″ having an oblong channel for capturing an oblong sheath wire 70′. The embodiment allows sheath wire to be angularly aligned “clocked” with respect to catheter sheath 40″ and for angle A to be formed in an optimized and predetermined way with respect to heart 10.

FIG. 12 shows an alternative embodiment for the end of wire 70. A coil section 70″ is a helical coil that allows angle A to rotate with respect to the axis of catheter sheath 40 as coil section 70″ is variably moved through flexible section 41.

While the catheter device and related methods described herein constitute preferred embodiments of the invention, it is to be understood that the invention is not limited to these precise form of assemblies, and that changes may be made therein without departing from the scope and spirit of the invention as defined in the appended claims.

I claim:

1. A catheter delivery device comprising:
a catheter sheath having an outside diameter and an inner channel,
a capsule connected to a first end of said catheter sheath;
a delivery device connected to a second end of said catheter sheath;
said catheter sheath having a flexible section between said first end and said second end;
a guide wire extending through said inner channel from said delivery device and extending past said capsule;
a sheath wire within said inner channel, said sheath wire in fixed connection to an actuator of said delivery device and extending towards said capsule, said sheath wire having a straight section and an angled section; and,
wherein movement of said actuator causes said angled section to create angled movement of said capsule relative to said catheter sheath.

2. The catheter delivery device of claim 1, wherein said flexible section has a flexible section outside diameter less than said sheath outside diameter.

3. The catheter delivery device of claim 1, wherein said catheter sheath has a sheath material stiffness greater than a flexible section material stiffness of said flexible section.

4. The catheter delivery device of claim 1, wherein said inner cavity has a first channel capturing said guide wire and a second channel capturing said sheath wire.

5. A catheter delivery device comprising:
a tubular catheter sheath having an outside diameter and an inner cavity,
a capsule connected to a first end of said catheter sheath, said capsule containing a prosthetic heart valve;
a delivery device connected to a second end of said catheter sheath;
said catheter sheath having a flexible section between said first end and said second end;
a guide wire extending through said inner cavity from said delivery device and extending past said capsule;
a sheath wire within said inner cavity, said sheath wire connected to an actuator of said delivery device and extending towards said capsule, said sheath wire having a straight section and a coil section; and,
wherein movement of said actuator causes said coil section to create rotating movement of said capsule relative to said sheath.

6. The catheter delivery device of claim 5, wherein said flexible section has a flexible section outside diameter less than said sheath outside diameter.

7. The catheter delivery device of claim 5, wherein said catheter sheath has a sheath material stiffness greater than a flexible section material stiffness of said flexible section.

8. The catheter delivery device of claim 5, wherein said inner cavity has a first channel capturing said guide wire and a second channel capturing said sheath wire.

9. A catheter delivery device comprising:
a catheter sheath having an outside diameter and a first channel and a second channel,
a capsule connected to a first end of said catheter sheath, said capsule containing a prosthetic heart valve;
a delivery device connected to a second end of said catheter sheath;
said catheter sheath having a flexible section between said first end and said second end;
a guide wire extending through said first channel from said delivery device and extending past said capsule;
a sheath wire within said second cavity, said sheath wire in fixed connection to an actuator of said delivery device and extending towards said capsule, said sheath wire having a straight section, a bend, and an angled section; and,
wherein movement of said actuator causes said angled section to be in contact with said capsule, said bend to be within said flexible section, and an angle to be formed between said capsule and said catheter sheath.

10. The catheter delivery device of claim 9, wherein said flexible section has a flexible section outside diameter less than said sheath outside diameter.

11. The catheter delivery device of claim 9, wherein said catheter sheath has a sheath material stiffness greater than a flexible section material stiffness of said flexible section.
